(19) United States
(12) Patent Application Publication
(10) Pub. No.: US 2014/0255379 A1
(43) Pub. Date: Sep. 11, 2014

(54) STABILIZED COMPOSITIONS AGAINST IONISING RADIATION

(71) Applicants: Paul Davis, Sharnbrook (GB); Jan Jezek, Cambridge (GB); Andrew Austin, Great Addington Northamptonshire (GB)

(72) Inventors: Paul Davis, Sharnbrook (GB); Jan Jezek, Cambridge (GB); Andrew Austin, Great Addington Northamptonshire (GB)

(73) Assignee: Microarray Limited, Sharnbrook, Bedfordshire (GB)

(21) Appl. No.: 14/238,532

(22) PCT Filed: Aug. 10, 2012
(86) PCT No.: PCT/GB2012/051944

§ 371 Date: May 7, 2014

(30) Foreign Application Priority Data
Aug. 12, 2011 (GB) 1113880.7

(51) Int. Cl. A61K 47/22; A61K 47/20; A61K 47/14; A61B 5/145; A61B 5/1468; A61K 38/44; A61K 47/18

(52) U.S. Cl. CPC A61K 47/22; A61K 38/443; A61K 47/20; A61K 47/183; A61B 5/14546; A61B 5/1468; A61K 47/14
USPC 424/94.4; 422/22; 600/309

(57) ABSTRACT

This invention relates to improvements to medical devices such as biosensors containing proteins such as oxidoreductases, for example oxidase and/or peroxidase enzymes. More generally it relates to novel compositions containing proteins which are stabilised to ionising radiation.

This invention relates to improvements to medical devices such as biosensors containing proteins such as oxidoreductases, for example oxidase and/or peroxidase enzymes. More generally it relates to novel compositions containing proteins which are stabilised to ionising radiation.

### BACKGROUND TO THE INVENTION

Products in the healthcare market, particularly products involving contact with broken skin such as wound dressings, require sterilisation to avoid being a source of infection to the individual being treated. Other products that fall into this category include biosensors which may be used to provide readings concerning the contents of biological fluids such as wound exudates. In certain instances these biosensors are integrated into composite wound dressings although they may be a separate article of manufacture.

Advanced biosensors typically contain enzymes which catalyse reactions involved in a diagnostic or reporting step. Whereas it would be appropriate to sterilise such biosensors using ionising radiation, unfortunately many proteins in an aqueous environment exhibit a high sensitivity to ionising radiation leading to loss of enzymatic activity. Thus, sterilising protein containing biosensors using ionising radiation is a challenge.

The use of organic additives to stabilise proteins from the adverse effect of ionising radiation in other contexts has been described. For example, WO03/026704 (Clearant) describes the use of a multitude of organic substances to stabilise biological material such as bone products and collagen as well as anti-insulin monoclonal immunoglobulin and Factor VIII. Ascorbate, which is a strong reducing agent, appears to be particularly favoured. In general the issues involved in stabilising biological materials are not the same as those involving stabilising purified proteins. Moreover biosensors typically contain reagents in addition to proteins which have to be compatible with the stabilising additives. For example, components of biosensors commonly rely on redox reactions which rules out the inclusion of strong oxidising or reducing agents as stabilising additives. WO2007/034198 (Insense) discloses the use of substances which are hydroxyl radical quenchers for stabilising proteins in an aqueous environment.

A specific type of biosensor of interest in the art is a lactate biosensor which can report on the level of lactate present in wound exudate. Recent developments have shown that localised lactate production in wounds reflects processes and conditions that affect wound healing. High levels of lactate (e.g. above 18 mM) are known to be deleterious to wound healing whilst levels around 3 mM are helpful. In one embodiment of a lactate biosensor, the presence of lactate in a secretion is detected by use of lactate oxidase to oxidise lactate and produce a stoichiometric amount of hydrogen peroxide which in turn triggers an electrochemical or a colorimetric indicator. In the context of a biosensor whose purpose is to detect lactate, lactate is obviously ruled out as a stabilising additive.

The solutions to problems of stabilising biosensors may be expected to have application more generally in stabilising other protein containing compositions.

We have now invented an improvement to biosensors and other protein containing compositions thereby to facilitate their stabilisation to ionising radiation with improved efficacy, convenience or compatibility as compared with the prior art.

### SUMMARY OF THE INVENTION

According to a first aspect of the invention there is provided an aqueous composition comprising one or more substantially pure proteins and comprising two or more protective substances, the first protective substance being methionine or the anion of an organic carboxylic acid not being an amino acid having a rate of reaction with hydroxyl radicals of greater than 10⁹ Lmol ⁻¹s⁻¹ and the second protective substance, different to the first protective substance, being selected from the list consisting of aromatic amino acids, nicotinate, purine, methionine and malate.

According to a second aspect of the invention there is provided an aqueous composition for use in a medical device such as a lactate biosensor comprising one or more substantially pure proteins and comprising two or more protective substances, the first protective substance being methionine or the anion of an organic carboxylic acid not being an amino acid or lactate and having a rate of reaction with hydroxyl radicals of greater than 10⁹ Lmol ⁻¹s⁻¹ and the second protective substance, different to the first protective substance, being selected from the list consisting of aromatic amino acids, nicotinate, purine, methionine and malate.

According to a third aspect of the invention there is provided a medical device such as a lactate biosensor comprising an aqueous composition according to the second aspect of the invention.

According to a fourth aspect of the invention there is provided a process for sterilising a composition or device which comprises irradiating a composition or device according to the invention with ionising radiation.

According to a fifth aspect of the invention there is provided a method of stabilising an aqueous composition comprising one or more substantially pure proteins and comprising two or more protective substances according to an aspect of the invention described herein, or a medical device such as a biosensor containing it, which comprises exposing said composition or device to ionising radiation. The one or more substantially pure proteins are protected from experiencing loss in activity as compared with a similar composition not containing the two or more protective substances.

Other aspects of the invention will be apparent from the foregoing.

### BRIEF DESCRIPTION OF THE FIGURE

FIG. 1 shows an example biosensor according to an aspect of the invention.

### DETAILED DESCRIPTION OF THE INVENTION

In one embodiment of the invention, the composition contains the anion of an organic carboxylic acid not being an amino acid having a rate of reaction with hydroxyl radicals of greater than 10⁹ Lmol ⁻¹s⁻¹ as the first protective substance. In such compositions of the invention the organic carboxylic acid as first protective substance may be a monocarboxylic acid. Alternatively it may be a dicarboxylic acid. The organic carboxylic acid may, for example, be selected from the group consisting of lactic acid, nicotinic acid, malic acid, benzoic acid, cinnamic acid, folic acid, salicylic acid and phthalic acid. For use in a lactate biosensor the first carboxylic acid is not lactic acid.

The rate of reaction with hydroxyl radicals of a substance may for example be measured by pulsed radiolysis under the conditions described in Buxton G. V, Greenstock C. L, Heiman W. P. and Ross A. B. J. Phys. Chem. Ref. Data. 17: 513-886 (1988).

In one embodiment of the invention, the composition contains lactate as first protective compound. In another embodiment of the invention, the composition contains nicotinate as first protective compound. In another embodiment of the invention, the composition contains malate as first protective compound.

Suitably the first protective substance has a rate of reaction with hydroxyl radicals of greater than 10¹⁰ Lmol ⁻¹s⁻¹.

In one embodiment the first protective substance is methionine. Mixtures of enantiomers including racemic mixtures may be used, or a single enantiomeric form may be used, especially the L enantiomer.

Suitably the first protective substance is not a strong reducing agent such as ascorbate or an organic carboxylic acid with a free thiol group. Suitably the first protective substance is not an oxidising agent, for example suitably it is incapable of oxidising iodide to iodine under the conditions of the particular application.

Suitably the concentration of the first protective compound is 5 to 200 mM and more suitably in the range 10 to 100 mM, e.g. 10 to 50 mM for example 10 mM, 25 mM or 50 mM.

In one embodiment of the invention, the composition comprises nicotinate as second protective compound.

In another embodiment of the invention, the composition comprises an aromatic amino acid as second protective compound. The aromatic amino acids that may be the second protective compound may, for example, be selected from phenylalanine, tryptophan and tyrosine. Mixtures of enantiomers including racemic mixtures may be used, or a single enantiomeric form may be used, especially the L enantiomer. In another embodiment of the invention, the composition contains tryptophan as second protective compound.

In another embodiment of the invention, the composition contains methionine as second protective compound.

Suitably the concentration of the second protective compound is 5 to 200 mM and more suitably in the range 10 to 100 mM, e.g. 10 to 50 mM for example 10 mM, 25 mM or 50 mM.

Thus a composition according to the invention may comprise (i) nicotinate and methionine; or (ii) lactate and phenylalanine; or (iii) nicotinate and tryptophan; or (iv) lactate and nicotinate; or (v) nicotinate and purine; or (vi) malate and nicotinate; or (vii) methionine and tryptophan.

In further embodiments, a composition according to the invention may comprise (viii) lactate and tryptophan; or (ix) nicotinate and phenylalanine; or (x) lactate and methionine; or (xi) methionine and an aromatic amino acid e.g. phenylalanine; or (xii) malate and phenylalanine; or (xiii) malate and tryptophan; or (xiv) lactate and tryptophan.

A composition according to the invention may also comprise a third different protective compound selected from the list consisting of aromatic amino acids, nicotinate, purine, methionine and malate.

Thus a composition according to the invention may contain (i) lactate, nicotinate and phenylalanine; or (ii) lactate, nicotinate and methionine; or (iii) nicotinate, tryptophan and phenylalanine; or (iv) nicotinate, tryptophan and methionine; or (v) nicotinate, methionine and purine; or (vi) nicotinate, methionine and phenylalanine; or (vii) lactate, methionine and purine or (viii) lactate, nicotinate and tryptophan; or (ix) lactate, tryptophan and purine; or (x) lactate, tryptophan and methionine as first, second and third protective compounds respectively.

Suitably the concentration of the third protective compound is 5 to 200 mM.

Suitably the pH of the composition is 4.5 to 8.5. For example, when the composition is a composition containing glucose oxidase the pH is suitably 4.5 to 5.5 e.g. around 5. When the composition is a composition containing lactose oxidase, the pH is suitably around 7 e.g. between pH 6 and 7. More generally the pH of the composition is within 1 pH unit e.g. within 0.5 pH unit of the pH at which a protein in the composition has maximum stability especially maximal thermal stability during storage. The storage stability is measured with respect to a stability aspect of the protein that is of critical importance for a specific application. For example, enzyme activity, measured by a suitable colorimetric or electrochemical method, may be a critical stability aspect for an enzyme used in a biosensor. In other applications, aggregation of a protein, measured by techniques such as size exclusion chromatography, differential light scattering or light obscuration techniques, may be the critical stability aspect for which optimal conditions are sought. In some cases, more than one stability aspect of a protein is critical for a particular application. The pH at which all critical aspects are best controlled is then sought, which thus becomes the pH of maximal thermal stability.

Suitably the one or more substantially pure proteins comprises or consists of an enzyme for example an oxidoreductase or peroxidase enzyme, for example glucose oxidase or lactate oxidase or horseradish peroxidase. The activity that is protected by the protective substances is the corresponding enzymatic activity. The protective substances may also protect the structural integrity of the enzymes, for example by preventing aggregation or formation of chemically modified variants of the enzymes.

By “substantially pure protein” is meant a protein isolated from other proteins with which it may be naturally associated e.g. isolated from biological material such as tissue, bone, body fluid etc. Alternatively it may be a protein produced by a recombinant process, e.g. obtained by expression in a cell-based system. Hence the protein content of the composition substantially consists or consists essentially of the one or more substantially pure proteins. For example the protein content of the composition is 95% or 98% or 99% or 99.5% or more composed of the one or more proteins (i.e. the protein content of the composition which does not consist of the one or more proteins is 5% or less or 2% or 1% or 0.5% or less).

In an embodiment of the invention there is provided a medical device comprising a composition according to the invention. For example, the medical device may be a wound dressing. The medical device may be a biosensor or a composite wound dressing that contains a biosensor. Alternatively the medical device may be one suitable for delivery of a therapeutic protein in a liquid composition, e.g. a prefilled syringe, an auto injector, a microneedle injector, a transdermal patch, an infusion pump, an inhalation device such as a nebuliser, a stent or an implant.

Ionising radiation that may be used to stabilise an aqueous composition or device according to the invention includes gamma radiation, electron beam radiation of X-ray radiation, especially gamma radiation. The total radiation dose may be at least 5 kGy, e.g. at least 15 kGy, e.g. at least 25 kGy e.g. at least 50 kGy. Preferably, the total radiation dose is between 25 to 50 kGy. Stabilisation with ionising radiation may be performed at a temperature of 4 to 40° C., for example 15 to 30° C. In an alternative embodiment it may be performed at low temperature e.g. below 4° C. such as below 0° C. such as below −20° C.

In one embodiment of the invention, of particular use in biosensor applications, the aqueous composition may be a hydrogel.

According to one preferred aspect of the invention there is provided a medical device which is a lactate biosensor, for use on a wound in the skin of a human or animal, the biosensor being sealed in packaging and comprising a sealed opening which, in use, the opening is exposed and placed over the wound site, the biosensor comprising a sensing means comprising an aqueous composition comprising lactate oxidase enzyme in hydrated condition together with two or more protective substances, the first protective substance being methionine or the anion of an organic carboxylic acid not being an amino acid or lactate and having a rate of reaction with hydroxyl radicals of greater than 10⁹ Lmol ⁻¹s⁻¹ and the second protective substance, different to the first protective substance, being selected from the list consisting of aromatic amino acids, nicotinate, purine, methionine and malate, and a hydrogen peroxide indicator means, the packaging being in contact with the indicator means and being transparent over a region of contact with the indicator means, the biosensor further comprising a means for preventing the ingress of any molecule larger than lactate into the sensing means, thereby allowing lactate to enter the biosensor from the wound, being oxidised to form hydrogen peroxide by the action of the lactate oxidase, the hydrogen peroxide thereby triggering the indicator means to indicate the presence of lactate in the wound.

The sensing means may suitably comprise a hydrated hydrogel. The lactate oxidase enzyme, the protective substances and the hydrogen peroxide indicator means may, for example, be dissolved in the water together with the swelling agent(s) to form the hydrogel. Suitable hydrogels are disclosed in WO03/090800 which is herein incorporated by reference in its entirety. A exemplary hydrogel comprises poly 2-acrylamido-2-methylpropane sulfonic acid or a salt thereof, preferably in an amount of about 20% by weight of the total weight of the gel.

Suitably the hydrogen peroxide indicator means comprises a peroxidase enzyme and a chromogenic material. Suitably the chromogenic material comprises iodide. Iodide is oxidised by hydrogen peroxide to iodine. In the presence of a complexing agent such as starch or polyvinyl acetate a wide range of bright colours can be generated to provide a visually perceptible indicator. The hydrogen peroxide indicator means may, for example, provide a visual indicator which is in proportion to the concentration of hydrogen peroxide (and thus lactate) in the sensing means.

When the hydrogen peroxide indicator means comprises iodide then the protective substances must be compatible with iodide and iodine for example they must not be capable of oxidising iodide to iodine or reducing iodine to iodide.

Suitably the means for preventing the ingress of molecules larger than lactate into the sensing means comprises a semi-permeable membrane. This prevents the biosensor from giving erroneous readings such as may be caused by other components that may be present in the fluid, especially catalase which is present in wounds.

Suitably the sensing means comprises a hydrated hydrogel and the means for preventing the ingress of molecules larger than lactate into the sensing means is provided by the hydrogel.

Suitably the lactate biosensor according to any one of the preceding claims, comprises a diffusion means situated between the sealed opening and the sensing means.

Suitably the biosensor comprises absorbent wick material which provides a fluid diffusion path from the opening to the sensing means. The absorbent wick material may, for example, comprise a fabric which is dry, partially dry or saturated with water.

In a further embodiment the sensing means can also comprise a control region which provides a visual indication of the flow of fluid into the sensing means of the biosensor. For example the sensing material may provide a visual indication of the presence of glucose. This particular region or a part of it may suitably contain glucose oxidase instead of lactate oxidase. A region upstream of the region containing glucose oxidase is suitably doped with glucose such that ingress of fluid (e.g. wound exudate) into the sensing means causes glucose to come into contact with glucose oxidase leading to production of hydrogen peroxide. This can be detected by means of the hydrogen peroxide indicator mentioned above.

In other embodiments, compositions of the invention may be used for pharmaceutical purposes for the treatment of humans and other animals. Hence the one or more substantially pure proteins contained in compositions of the invention may be therapeutic proteins including monoclonal antibodies hormones, and the like. Further examples of therapeutic proteins include therapeutic enzymes, blood coagulation factors, monoclonal antibody fragment, fusion proteins, cytokines and the like. Compositions for pharmaceutical purposes may also contain conventional additives including additives to modify tonicity, lyoprotectants, preservatives etc. Further additives include buffers, surfactants etc.

Compositions of the invention and medical devices containing them may have the advantage that the proteins they contain (or one or more of them) are relatively protected against damage (such as loss of activity, aggregation or chemical modification of the proteins) by ionising radiation used in sterilisation processes as compared with compositions lacking the protective substances. Activity of proteins may be measured using conventional processes. For example enzyme activities may be measured using chromogenic or electrochemical assays. Activity of lactate oxidase may be measured by following the rate of hydrogen peroxide production in a solution of lactate using a chromogenic assay or an electrochemical assay. Activity of glucose oxidase may be measured by following the rate of hydrogen peroxide production in a solution of glucose using a chromogenic assay or an electrochemical assay.

Activity of horseradish peroxidase may be measured by measuring the rate of oxidation of a suitable dye (such as tetramethylbenzidine) in the presence of excess hydrogen peroxide using a chromogenic assay.

The invention will be illustrated by reference to the following non-limiting examples.

### EXAMPLES

### Example 1

### Example Lactate Biosensor

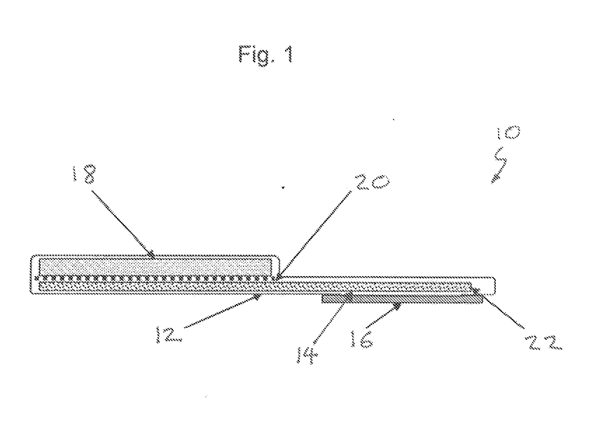

By reference to FIG. 1, a biosensor 10 is shown sealed in clear transparent packaging 12, and having an opening 14 covered by removable seal 16.

The biosensor 10 comprises a sensing means 18 which comprises a hydrated hydrogel containing lactate oxidase, horseradish peroxidase enzyme, two or more protective substances to protect the lactate oxidise and horse radish peroxidise enzymes during sterilisation, iodide as the chromogenic material, and starch.

The biosensor also comprises semi-permeable membrane 20 which allows the free passage of water, lactate and other low molecular weight solutes but prevents passage of high molecular weight solutes such as enzymes e.g. catalase.

The biosensor 10 also comprises an absorbent wick material 22 which provides a fluid diffusion path from the opening 14 to the sensing means 18 and comprises a fabric saturated with water, although many other versions are possible.

In use, the seal 16 is removed and the opening 14 is placed over a wound in the skin of a human or animal subject.

Wound exudates then diffuses into the biosensor through opening 14 and diffuses along the absorbent wick 22. Once at the semi-permeable membrane 20 only the lactate and other low molecular weight solutes continue to diffuse into the hydrogel 18.

Once in the hydrogel the lactate oxidase causes oxidation of the lactate to form hydrogen peroxide. The formed hydrogen peroxide then oxidises the iodide with the action of the peroxidase enzyme to form iodine. The iodine then complexes with the starch which forms a distinctive blue colour. This causes a visual indication in a change of colour of the hydrogel, which is visible through the clear transparent packaging.

### Example 2

### Sterilisation protocol

A biosensor device may be sterilised by means of a gamma-radiation service, of which there are many specialist commercial services to choose from (e.g. Isotron Ltd in the UK). The device is typically placed in an aluminium foil pouch and sealed. At the gamma radiation facility the pouch is placed in a radiation chamber for a period of time decided by a trained and qualified operator under conditions that ensure that the device receives a radiation dose of between 25 and 40 kGy.

### Example 3

### Effect of Selected Excipients of the Recovery of Aqueous Enzyme Activity of Glucose Oxidase Following Sterilisation by Gamma Radiation (25 kGy)

Aqueous solutions (1 ml) of glucose oxidase (350 pg/ml) were prepared with selected additives in 2 ml glass (Type I) vials and sealed with a crimp top. The vials were gamma-irradiated by an industrial sterilisation service (Isotron Ltd, Swindon, Wilts, UK) using a Cobalt 60 gamma source and a 25 kGy dose. The gamma-irradiated solutions were subsequently tested for glucose oxidase activity. This was performed according to the following procedure: 50 μL of the solution was added to 50 mL of deionised water. The following solutions were then added:
10 mL of reagent mix (5 parts of 0.1 M sodium phosphate, pH 6+4 parts 2% w/w starch+1 part of 1mg/mL lactoperoxidase enzyme);
5 mL of 100 mM potassium iodide and
5 mL of 40% w/w glucose solution.

These were mixed together quickly. Time =0 was counted from the addition of the glucose. After 5 min, 1 ml of 5 M aq. hydrochloric acid was added to stop the reaction. The absorbance was then read at 630 nm. If the colour intensity was too great to allow an accurate reading, the sample was diluted with a defined volume of deionised water to bring the colour back on scale. The results were expressed as percentage recovery, by reference to the absorbance measured in the pre-gamma irradiation samples.

The effect of a number of excipients in various combinations on the recovery of glucose oxidase activity was assessed. Results are shown in Table 1 below:

TABLE 1
Activity recovery of glucose oxidase in aqueous formulations
following gamma irradiation. All formulations were adjusted
to pH 5.0 and contained 50 mM NaCl.
Part 1

| Excipient | Alone | Lactate+ | Methionine+ | Nicotinate+ |
|---|---|---|---|---|
| No | 0 | 21 | 26 | 32 |
| excipients | | | | |
| Lactate | 21 | | | |
| (50 mM) | | | | |
| Nicotinate | 32 | 69 | 88 | |
| (50 mM) | | | | |
| Purine | 9 | 42 | 37 | 61 |
| (50 mM) | | | | |
| Tryptophan | 60 | 65 | 88 | 76 |
| (15 mM) | | | | |
| Phenylalanine | 25 | 88 | 38 | 46 |
| (25 mM) | | | | |
| Methionine | 26 | 51 | | |
| (50 mM) | | | | |
| Malate | 5 | 32 | 45 | 56 |
| (50 mM) | | | | |

Part 2

| | | | | Nico- | Nico- |
|---|---|---|---|---|---|
| | Lactate+ | Lactate+ | Lactate+ | tinate+ | tinate+ |
| | Nico- | Trypto- | Methi- | Trypto- | Methi- |
| Excipient | tinate+ | phan+ | onine+ | phan+ | onine+ |
| No | 69 | 65 | 51 | 76 | 88 |
| excipients | | | | | |
| Lactate | | | | | |
| (50 mM) | | | | | |
| Nicotinate | | | | | |
| (50 mM) | | | | | |
| Purine | 68 | 96 | 99 | 79 | 101 |
| (50 mM) | | | | | |
| Tryptophan | 94 | | | | |
| (15 mM) | | | | | |
| Phenylalanine | 102 | 78 | 78 | 99 | 100 |
| (25 mM) | | | | | |
| Methionine | 97 | 93 | | 102 | |
| (50 mM) | | | | | |
| Malate | | | | | |
| (50 mM) | | | | | |

As can be seen from Table 1, the compositions containing two protective substances were more effective than the compositions containing a single protective substances (i.e. inclusion of lactate, methionine or nicotinate improved the protective effect of compositions containing another single protective substance). The combinations of three protective substances were yet more effective.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications mentioned throughout the specification of the present invention are herein incorporated in their entirety by reference.

The invention embraces all combinations of preferred and more preferred groups and suitable and more suitable groups and embodiments of groups recited above.

1. A medical device comprising an aqueous composition lactate oxidase and two or more protective substances, the first protective substance being methionine or the anion of an organic carboxylic acid not being an amino acid having a rate of reaction with hydroxyl radicals of greater than 10^{9 Lmol} ⁻¹s⁻¹ and the second protective substance, different to the first protective substance, being selected from the list consisting of aromatic amino acids, nicotinate, purine, methionine and malate and wherein none of the protective substances is lactate.

2. A device according to claim 1 wherein the first protective substance is the anion of an organic carboxylic acid not being an amino acid having a rate of reaction with hydroxyl radicals of greater than 10^{9 Lmol} ⁻¹s⁻¹.

3. A device according to claim 2 wherein the organic carboxylic acid is a monocarboxylic acid.

4. A device according to claim 2 wherein the organic carboxylic acid is selected from the group consisting of nicotinic acid, malic acid, benzoic acid, cinnamic acid, folic acid, salicylic acid and phthalic acid.

5. A device according to claim 1 wherein the concentration of the first protective compound is 5 to 200 mM.

6. A device composition-according to claim 1 wherein the concentration of the second protective compound is 5 to 200 mM.

7. (canceled)

8. A device according to claim 1 which contains nicotinate as first protective compound or as second protective compound.

9. (canceled)

10. A device according to claim 1 which contains tryptophan as second protective compound.

11. A device according to claim 1 which contains methionine as second protective compound.

12. A device according to claim 1 which comprises (i) nicotinate and methionine; or (ii) nicotinate and tryptophan; or (iii) nicotinate and purine; or (iv) malate and nicotinate; or (v) methionine and tryptophan.

13. A device according to claim 1 which comprises (i) nicotinate and phenylalanine; or (ii) methionine and an aromatic amino acid; or (iii) malate and phenylalanine; or (iv) malate and tryptophan.

14. A device according to claim 1 comprising a third different protective compound selected from the list consisting of aromatic amino acids, nicotinate, purine, methionine and malate.

15. A device according to claim 14 which contains (i) nicotinate, tryptophan and phenylalanine; or (ii) nicotinate, tryptophan and methionine; or (iii) nicotinate, methionine and purine; or (iv) nicotinate, methionine and phenylalanine; as first, second and third protective compounds respectively.

16. A device according to claims 1 wherein the pH of the composition is 4.5 to 8.5.

17-20. (canceled)

21. A device according to claim 16 wherein the pH of the composition is 6 to 7.

22-23. (canceled)

24. A medical device according to claim 1 which is a wound dressing.

25. A medical device according to claim 1 which is a lactate biosensor.

26. A medical device which is a lactate biosensor for use on a wound in the skin of a human or animal, the biosensor being sealed in packaging and comprising a sealed opening which, in use, the opening is exposed and placed over the wound site, the biosensor comprising a sensing means comprising an aqueous composition comprising lactate oxidase enzyme in hydrated condition together with two or more protective substances, the first protective substance being the anion of an organic carboxylic acid not being an amino acid or lactate and having a rate of reaction with hydroxyl radicals of greater than 10⁹ Lmol ⁻¹s⁻¹ and the second protective substance, different to the first protective substance, being selected from the list consisting of aromatic amino acids, nicotinate, purine, methionine and malate, and a hydrogen peroxide indicator means, the packaging being in contact with the indicator means and being transparent over a region of contact with the indicator means, the biosensor further comprising a means for preventing the ingress of any molecule larger than lactate into the sensing means, thereby allowing lactate to enter the biosensor from the wound, being oxidised to form hydrogen peroxide by the action of the lactate oxidase, the hydrogen peroxide thereby triggering the indicator means to indicate the presence of lactate in the wound.

27. A process for sterilising a composition or device which comprises irradiating a device according to claim 1 with ionising radiation.

28. A device according to claim 1 which is sterilised by irradiation with ionising radiation.
